(19) United States
(12) Patent Application Publication
(10) Pub. No.: US 2016/0228013 A1
(43) Pub. Date: Aug. 11, 2016

(54) TRANSCATHETER AORTIC VALVE IMPLANTATION PRESSURE WIRES AND USES THEREOF

(71) Applicant: CEDARS-SINAI MEDICAL CENTER, Los Angeles, CA (US)

(72) Inventors: Hasanian Al-Jilaihawi, West Hollywood, CA (US); Rajendra Makkar, Los Angeles, CA (US)

(73) Assignee: Cedars-Sinai Medical Center, Los Angeles, CA (US)

(21) Appl. No.: 15/022,874

(22) PCT Filed: Oct. 7, 2014
(86) PCT No.: PCT/US14/59547

§ 371 Date: Mar. 17, 2016

Related U.S. Application Data
(60) Provisional application No. 61/920,363, filed on Dec. 23, 2013.
(60) Provisional application No. 61/887,816, filed on Oct. 7, 2013.

(51) Int. Cl. A61B 5/0215; A61B 5/0402; A61B 5/00

(52) U.S. Cl. CPC A61B 5/0215; A61B 5/6851; A61B 5/0402; A61B 5/6852; A61B 5/742; A61B 2560/0223

(57) ABSTRACT

Described herein is a guide wire that includes one, two or multiple pressure transducers for use in TAVI. The guide wire may include an aortic pressure sensor spaced from a left ventricular pressure sensor with sufficient length to allow the aortic pressure sensor to be located in the aorta while the ventricular pressure sensor is simultaneously located in the left ventricle. The pressure readings between the left ventricle and aorta may be subtracted to determine an improved indication of the prognosis of a patient with intermediate post-TAVR aortic regurgitation after assessment with transesophageal echocardiography.

### FIELD OF INVENTION

The present invention is directed to guide wires for sensing pressures and methods of using the same.

### BACKGROUND OF THE INVENTION

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Transesophageal echocardiography (TEE) is presently the modality of choice in the comprehensive peri-procedural assessment of post-TAVR aortic regurgitation (AR) and can evaluate both severity and mechanism, distinguishing valvular from paravalvular (PV) AR. It is established that PV AR is associated with increased mortality after TAVR. However, the quantification of PV AR can be difficult, particularly in the intermediate range of severity, such that the survival of mild and moderate-severe PV AR were similar in the PARTNER trial. Recently, the aortic regurgitation index (ARi), studied principally after self-expanding TAVR, has offered incremental value to angiographic assessment in the risk stratification of PV AR. Its additional value to the TEE assessment of post TAVR AR has not been demonstrated, nor has it been applied systematically to balloon expandable TAVR. Moreover, it is known that heart rate can influence diastolic transcatheter hemodynamics and can therefore dramatically alter the ARi. The inventors sought to better understand transcatheter hemodynamic data in the setting of post TAVR AR and how it may be best integrated into clinical practice and decision making for further therapy.

### SUMMARY OF THE INVENTION

The following embodiments and aspects thereof are described and illustrated in conjunction with systems, compositions and methods which are meant to be exemplary and illustrative, not limiting in scope.

Described herein is a pressure sensing wire assembly for measuring pressure in the heart of a patient that has undergone or is undergoing TAVI. In some embodiments, the assembly may include a guide wire, an aortic pressure sensor, a ventricular pressure sensor and an interface on a distal end of the guide wire to communicate signals from the pressure sensors. The guide wire may be insertable into a heart and the aortic pressure sensor and ventricular pressure sensor may be spaced the appropriate distance from each other in order for the aortic section to be located in the aorta of the heart while the ventricle section is located in a ventricle of the heart. Accordingly, in some embodiments, the guide wire may contain mechanical or electrical mechanisms to shorten or lengthen the distance between the aortic pressure sensor and ventricular pressure to accommodate different sized hearts, for example a child versus adult heart. Additionally, the guide wire must be of sufficient overall length for the sensors disclosed to reach the heart from the insertion point into the body (e.g., the femoral artery, etc.). In some embodiments, the aortic pressure sensor on the aortic section of the guide wire senses the pressure in the aorta. In some embodiments, the ventricle pressure sensor on the ventricle section of the guide wire senses the pressure in the ventricle (for example in the left ventricle).

Also described herein is a method that includes providing a subject that is undergoing or has undergone TAVI, obtaining a transesophageal echocardiogram to determine aortic regurgitation, and whether, for example, aortic regurgitation is low, intermediate (mild or moderate) or severe, and then determining a heart rate adjusted diastolic delta in the subject. In some embodiments, the heart rate adjusted diastolic delta will only be determined if the subject has intermediate aortic regurgitation. A heart rate adjusted diastolic delta of less than or equal to the reference value is indicative of poor prognosis in the subject and the heart rate adjusted diastolic delta of greater than the reference value is indicative of good prognosis in the subject.

Disclosed also is a method of manufacturing a guide wire and or catheter combination with an aortic pressure sensor, a ventricular pressure sensor and an interface on a distal end of the guide wire to communicate signals from the pressure sensors. This may include assembly of various components into the guide wire, including the addition of piezoresistors or other pressure sensors on the guide wire.

### BRIEF DESCRIPTION OF FIGURES

Exemplary embodiments are illustrated in the referenced figures. It is intended that the embodiments and figures disclosed herein are to be considered illustrative rather than restrictive.

FIGS. 1A-1B depict, in accordance with various embodiments of the present invention, (A) a cross-section view showing a sensor wire assembly with pressure sensors inserted in a heart prior to the implantation of a valve and (B) a cross-section view showing the sensor wire assembly with pressure sensors after the valve is implanted.

FIG. 2 depicts, in accordance with various embodiments of the present invention, a block diagram of the sensor system to measure pressure from the heart using the wire assembly in FIG. 1A.

FIG. 3 depicts, in accordance with various embodiments of the present invention, a block diagram showing the electronic components of the sensor of the wire assembly and a sensor system.

FIG. 4 depicts, in accordance with various embodiments of the present invention, a block diagram of an alternative sensor system to measure pressure from the heart using the wire assembly in FIG. 1A employing wireless communication.

FIG. 5 depicts, in accordance with various embodiments of the present invention a cross-section view of the interface unit for the sensor system in FIG. 4.

FIGS. 6A-6B depict, in accordance with various embodiments of the present invention, a comparison of the Bonn CAI score and the Los Angeles CHAI score. There are two principal differences. Firstly, stratification of moderate AR is not performed with the CAI score, whereas the CHAI score does not mandate intervention with moderate AR which is not hemodynamically significant. Secondly, the CAI score does not adjust for heart rate, whereas the CHAI score does.

FIG. 7 depicts, in accordance with various embodiments of the present invention, Kaplan-Meier survival curves compared by data available immediately post TAVR in the form of PV AR grade by TEE, graded by VARC 2 criteria. Survival to 1-year follow-up is shown.

FIGS. 8A-8B depict, in accordance with various embodiments of the present invention, the influence of bradycardia on key transcatheter hemodynamic parameters (ARi and HRA-DD); ARi—Aortic regurgitation index and HRA-DD—heart-rate adjusted diastolic delta.

FIGS. 9A-9D depict, in accordance with various embodiments of the present invention, Kaplan-Meier survival curves according to immediate post TAVR hemodynamic data. Heart rate adjustment improves the stratification of survival by DD but not the ARi.

FIGS. 10A.1-A.4, 10B.1-B.4 and C depict, in accordance with various embodiments of the present invention, the profound influence of heart rate on the ARi that can only be standardized with heart rate adjustment. The relevance of the composite echocardiographic-hemodynamic assessment in transcatheter valve-in-valve (TV-in-TV) for severe paravalvular AR due to malpositioning is shown. TEE (A.1 and B.1) demonstrated severe AR before the second valve was implanted (A.1) that resolved to mild AR immediately post TAVR (B.1). Heart rate was modified using ventricular pacing. Hemodynamic data (A.2-A.4 and B.2-B.4) is shown pre (A.2-A.4) and post TV-in-TV (B.2-B.4) for heart rates increased with ventricular pacing in the same patient. Data for the diastolic delta (AoDBP-LVEDP) is shown in this patient (patient 1) and 9 other cases that had transcatheter hemodynamics recorded during incremental transvenous pacing (10C). The diastolic delta increases in a linear fashion with heart rate but the slopes of the line differ, in some cases widely.

FIG. 11 depicts, in accordance with various embodiments of the present invention, ROC curves comparing the discrimination of 1-year mortality by TEE PV AR grade, Bonn CAI score and Los Angeles CHAI score.

FIGS. 12A-B depict, in accordance with various embodiments of the present invention, Kaplan-Meier curves stratifying survival by Bonn CAI score and Los Angeles CHAI score.

FIGS. 13A-13D depict, in accordance with various embodiments of the present invention, hemodynamic pressures and heart rate. Changes in immediate post TAVR transcatheter hemodynamic pressures with increasing heart rate altered by transvenous ventricular pacing are shown.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein, “AoDBP” is the aortic diastolic blood pressure.

As used herein, “AR/AI” is the aortic regurgitation/aortic insufficiency.

As used herein, “ARi” is the aortic regurgitation index.

As used herein, “AoSBP” is the aortic systolic blood pressure.

As used herein, “CHAI score” is the composite heart-rate adjusted hemodynamic-echocardiographic aortic insufficiency score. Specifically, the CHAI score is an ordinal value obtained through a combination of the data provided by the TEE and the transcatheter hemodynamic data adjusted to heart rate, as described herein.

As used herein, DD is the diastolic delta.

As used herein, HR is the heart rate.

As used herein, “HR-ARi” is the heart rate-adjusted aortic regurgitation index.

As used herein, “HRA-DD” is the heart rate-adjusted diastolic delta.

As used herein, LVEDP is the left ventricular end-diastolic pressure.

As used herein, “PV AR” is the paravalvular aortic regurgitation.

As used herein, “TAVR” is transcatheter Aortic Valve Replacement and is used interchangeably with transcatheter aortic valve implantation (TAVI).

As used herein, “TEE” is the transesophageal echocardiography.

Current methods for monitoring myocardial blood pressure during TAVI include insertion of a catheter comprising a pressure sensor into the aorta and insertion of a second catheter also comprising a pressure sensor into the left ventricle. The second catheter is inserted over a guide wire, advanced into the left ventricle and the guide wire is removed to obtain a pressure reading from the left ventricle. The disadvantage of this approach is that there is no ability to immediately assess hemodynamic pressures without removing the wire, which will create additional delay in important maneuvers that can be guided by the hemodynamic pressure readings, including valve post-dilatation and transcatheter valve-in-valve. Therefore there is a need in the art for a single device that measures the aortic and ventricular blood pressure simultaneously during the TAVI procedure or immediately after the TAVI procedure without removing the wire inserted for the TAVI procedure. Described herein is a guide wire suitable for TAVI that comprises two pressure sensors and uses of said guide wire to determine the prognosis of a subject that is undergoing TAVI or has undergone TAVI. In other embodiments, it is conceivable that a catheter with two pressure sensors may be utilized.

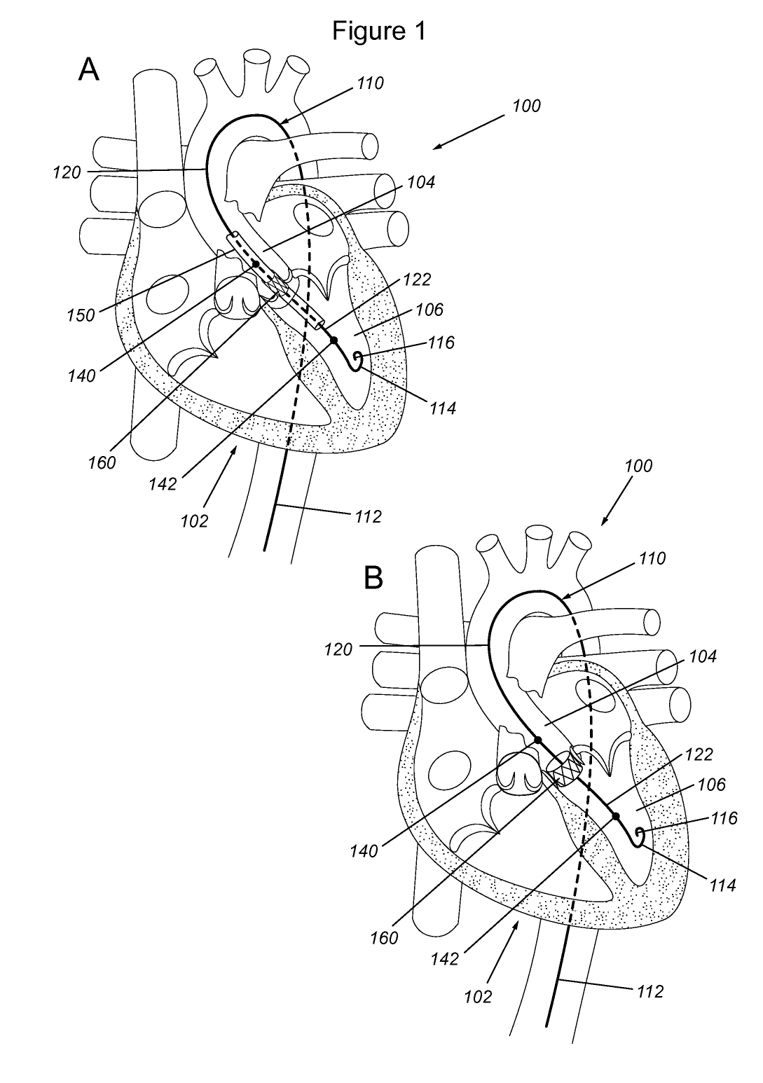

FIG. 1A is a cross section view of the components of the heart region 100 of a patient that may be undergoing trans-catheter aortic valve implantation or a similar procedure. The region 100 includes the heart 102 which has the aorta 104 and a left ventricle 106. Broadly, the implantation procedure includes the insertion of a guide wire 110 through the aorta 104 and a catheter which is used to replace the diseased valve with a replacement valve (not shown). The guide wire 110 is advanced through the aorta 104 and into the left ventricle 106. The guide wire 110 includes a proximal end 112 and an opposite distal end 114. The distal end 114 of the guide wire 110 includes a spiral shaped end 116. The spiral shaped end 116 provides a safety mechanism to protect against causing trauma on the vessel walls and other internal body structures, and is therefore less likely to cause ventricular perforation, a recognized problem potentially caused by the wire during TAVI. Accordingly, the rounded and blunt and that the spiral shaped end 116 creates, prevents the end of a guide wire from piercing the ventricle during a contraction of the ventricular muscle wall.

In some embodiments, the guide wire 110 is divided into an aortic section 120 which is closer to the proximal end 112 and a ventricle section 122 which is closer to the distal end 114. An aortic pressure sensor 140 is located in the aortic section 120 and a ventricle pressure sensor 142 is located in the ventricle section 122. The distance between the aortic pressure sensor and the ventricular pressure sensor may be designed so that the aortic pressure sensor 140 is located in the aorta at the same time that the ventricular pressure sensor 142 is in the left ventricle. This distance may take into account different sized subjects which have different sized hearts and the shrinkage of the ventricle during contraction. Accordingly, there may be window of distances between the aortic pressure sensor 140 and ventricular pressure sensor 142 in which they can be located in the aorta and left ventricle respectively at the same time. In other embodiments, the distance between the two pressure sensors may need to be adjustable to accommodate for different sized subjects. In some embodiments, the distance may be adjustable while the guide wire 110 is inserted into the subject. In some embodiments, the distance between the two sensors may be X″, Y″ or other suitable distances. The adjustment mechanism may include a second guide wire 110 wrapped around the first guide wire 110, it may include a catheter that has one pressure sensor and a guide wire that has a second pressure sensor. In other embodiments, it may include a thinner wire with a pressure sensor in a lumen of a larger catheter.

In some embodiments, the guide wire 110 has an outer diameter of approximately 0.035″, but other appropriate dimensions may be used such as 0.038″. The wire may be made of nitinol which will preserve its shape in vivo. In some embodiments, the pressure sensors for use with the pressure wire for use in TAVI described herein may be obtained from, for example, the Volcano Corporation, Radi Medical Systems and/or St. Jude Medical, Inc.

In some embodiments, the guide wire 110 is advanced into the heart 102 until the pressure sensor 140 of the aortic section 120 is in the aorta 104 and the pressure sensor 142 of the ventricle section 122 is in the left ventricle 106. In other embodiments, once the aortic pressure sensor 140 is in the aorta, the guide wire 110 may be adjusted to move the ventricular pressure sensor 142 into position in the left ventricle 106.

After the guide wire 110 is correctly positioned, a catheter 150 carrying a replacement valve 160 is inserted over the guide wire 110 as shown in FIG. 1B. FIG. 1B shows the placement of the valve 160 between the aorta 104 and the left ventricle 106, replacing the diseased aortic valve. Since the guide wire 110 is still in place, the aortic sensor 140 and the ventricle sensor 142 may provide hemodynamic pressure data from the aorta 104 and left ventricle 106 immediately after placement of the valve 160 or within a short time frame including 5 seconds, 10 seconds, 30 seconds, 2 minutes or other time frames. In some embodiments, the aortic pressure sensor 140 may be located on the catheter 150 so that when it is removed and the replacement valve 160 is installed the pressure sensor 140 may be positioned appropriately in the aorta. Accordingly, the guide wire 110 would not have to be adjustable in this embodiment to accommodate for different sized subjects.

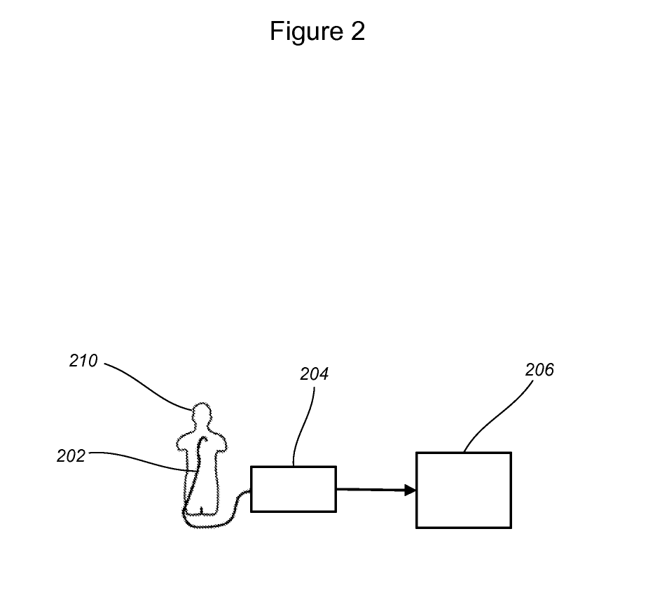

FIG. 2 is a block diagram of a pressure monitoring system 200 incorporating the elements shown in the region 100 in FIG. 1A. The pressure monitoring system 200 includes a pressure sensor wire assembly 202 which includes the guide wire 110 and pressure sensors 140 and 142 in FIG. 1A. The signals output from the pressure sensors 140 and 142 representing the pressure of the aorta and left ventricle are communicated to a sensor interface unit 204 where they undergo signals processing and conditioning. The resulting processed signals are then sent to an external physiology monitor 206. The external monitor 206 allows comparison of simultaneous pressures in the left ventricle 106 and aorta 104 to further stratify the severity of paravalvular regurgitation after the implantation of the transcatheter valve 150 in the heart 102 in FIG. 1 of a patient 210 as will be explained below. For instance, the external physiology monitor 206 or circuitry prior to the signals arriving at the external physiology monitor 206 may subtract the aortic pressure from the ventricular pressure. Then, the resultant difference may be displayed in various indications on a display associated with the external physiology monitor 206. For example, a numeric value of the pressure difference may be displayed, or an index based on the pressure difference. In other embodiments, the difference may be further utilized to indicate whether or not correct action needs to be immediately taken. This may be color coded or by other message. In some embodiments, the difference may be performed utilizing more simple electronic components such as a comparators and an LED or other simple display to determine whether the difference is above or below a set threshold (for example an index of 25 as disclosed herein) and whether corrective intervention needs to be taken.

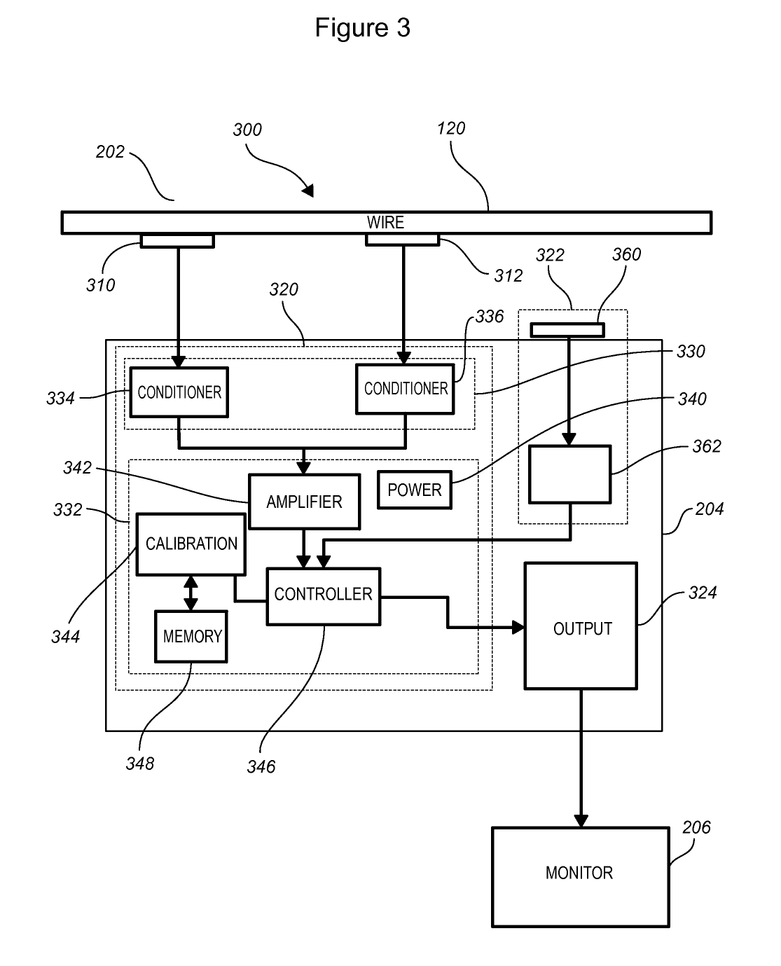

FIG. 3 is a block diagram of the electronic components of an example electrical system 300 of the pressure monitoring system 200 in FIG. 2. The electrical system 300 processes signals relating to or representing pressures measured from inside or near the heart of the patient 210 in FIG. 2 to measure hemodynamic pressure in the aorta and the left ventricle during or after the implantation procedure. In some embodiments, the pressure sensing wire assembly 202 measures pressure inside the left ventricle 106 and aorta 104 of the patient. The assembly 202 includes the guide wire 110 as described above. In some embodiments, the guide wire 110 includes a proximal pressure sensor circuit 310 which is included in the aortic pressure sensor 140 and a distal pressure sensor circuit 312 included in the ventricle pressure sensor 142 for measuring pressure in the aorta and the left ventricle respectively. The two sensor circuits 310 and 312 each generate a pressure sensor signal output in response to the sensed pressures. As explained above, the pressure sensor wire 110 includes the aortic pressure sensor 140 and the ventricle pressure sensor 142 and is adapted to be inserted into the heart of the patient 210 in order to position the sensor circuits 310 and 312 within the aorta 104 and left ventricle 106 respectively.

In some embodiments, the pressure sensors 140 and 142 each contain four piezoresistors embedded in a thin, chemically-etched silicon diaphragm. The piezoresistors are wired in a bridge circuit forming the sensor circuits 310 and 312. A pressure change causes the diaphragm to flex, inducing a stress in the diaphragm and the embedded resistors. The resistor values change in proportion to the stress applied and produce an electrical output. Accordingly, the voltage output by the piezoresistor will be proportional to the stress applied through the pressure of the aorta and left ventricle. Accordingly, by processing the voltage output from the piezoresistors, a value representative of the stress and therefore pressure inside the aorta and/or the left ventricle may be calculated. In other embodiments, other suitable pressure sensors may be utilized including capacitive, interferometric sensors, and others.

The sensor interface unit 204 includes modules which receive the pressure sensor signals output from the pressure sensor circuits 310 and 312. These signals may be output in analog or digital form and are sent to the sensor interface unit 204, where they can be conditioned and processed for analysis. For example, the sensor interface unit 204 may include various components, including an analog to digital converter to convert the signals into digital data, which then may be analyzed or output in data packets in a standardized or specialized format for a specific external physiology monitor 206. The interface unit 204 includes a sensor signal adaption module 320, a pressure compensation module 322 and an output interface 324. The sensor signal adaption module 320 includes a programmable sensor conditioning unit 330 and a calibration unit 332. The sensor conditioning unit 330 includes a sensor conditioner 334 coupled to the output of the aortic sensor circuit 310 and a sensor conditioner 336 coupled to the output of the ventricle sensor circuit 312. Both conditioners 334 and 336 may include various filters, A/D converters, signal level adjustments, and other signal conditioning components. For example, the conditioners 334 and 336 may include various noise filters, notch filters to isolate the relevant pressure signals, and other components.

The calibration unit 332 includes a power source 340, an output amplifier circuit 342, a calibration circuit 344, a microcontroller 346, and a storage device 348. The storage device 348 allows calibration data to be supplied, stored and altered. In this example, the storage device 348 is an electrically erasable programmable read-only memory (EEPROM). Of course other storage devices such as read only memory (ROM), random access memory (RAM), flash memory, etc. may be used for the storage device 348.

The sensor signal adaption module 320 receives signals from the pressure circuits 310 and 312 and conditions and processes them for sending to the external physiological monitor 206. For instance, in some embodiments, the adaptation module 320 will receive analog signals from the pressure circuits 310 and 312. Those signals may be first filtered using analog filters and then converted to digital signals using an A/D converter. In other embodiments, the signals may first be converted and then digital filters may be applied, or both. In some embodiments, the signals may also be amplified 342.

Then, the system may analyze and process the digital signals to determine the pressure readings from the signals output from the pressure circuits 310 and 312. To do this, for example, the controller 346 may apply algorithms that convert the voltage into pressure readings based on a known correlation between the voltage and other features of the signals output from the pressure circuits 310 and 312. For example, standard equations may be utilized to determine pressure from the signals output from piezoelectric resistors or other pressure sensing components. For example, the Voltage from a piezoelectric material can generally be calculated from the following equation:

V=Sᵥ*P*D (1)

Where V=Piezoelectric generated voltage (Volts), Sᵥ=voltage sensitivity of the material, P=pressure, and D=thickness of the material. Accordingly, with calibration data for individual piezoresistors a direct correlation between output voltage and sensed pressure can be determined and stored in the system. The pressure readings may then be sent as standard data packets using a standard instrumentation protocol or specialized for a specific physiological monitor by controller 346 or other control system in communication with the sensor interface unit 204. The data packets or digital data in other formats may then be sent to an external monitor 206 via a direct connection, or over a network to the monitor 206. The monitor 206 may then display the pressure readings or perform further calculations on the pressure readings as described further herein.

While determining the pressures from the signals output from sensor circuits 310 and 312, calibration data is utilized specific to each individual sensor circuit 310 and 312 to convert the voltage or other signal characteristics into pressure readings. Accordingly, in some embodiments, a calibration circuit 344 may be provided that provides calibration data for the controller 346 in order to convert the signals form the sensor circuits 310 and 312 into pressure readings, based on data recorded at the manufacturing plant or in a lab for each individual sensor circuit and pressure sensor 310 and 312. The memory device 348 contains individual calibration data obtained during calibration of the sensor circuits 310 and 312 performed for each individual sensor wire assembly such as the assembly 202. The calibration is performed in connection with manufacturing of the guide wire 110. Calibration data takes into account parameters such as voltage offsets and temperature drift, etc. and is stored in the memory device 348.

Power is delivered to pressure sensor circuits 310 and 312 from either the calibration circuit 344 via voltage generated by the calibration unit 332. As an alternative, the pressure sensor circuits 310 and 312 may be powered from a separate energy source, e.g. a battery or a capacitor, or from an external power supply, e.g. an external main supply via the monitor 206.

In this example, for a given excitation voltage applied to one of the sensor circuits such as the sensor circuit 310, the output voltage of the sensor circuits 310 and 312 is a voltage proportional to the pressure applied to the sensor 140. Hence, the sensor output voltage of the bridge circuit is proportional to the pressure applied to the sensor 140, which for a given pressure will vary with the applied voltage. The voltage output from the sensor circuits 310 and 312 is preferably compensated for temperature variation at the using filters or module integrated with the sensor circuits 310 and 312 and the compensated sensor output voltage is applied to the interface unit 204.

The controller 346, which may be a processor, a microprocessor, microcontroller, control system with multiple processors, or similar programmable device or control system as described further herein, and may further be employed to process and adapt the conditioned signals output from the sensor circuits 310 and 312. In some embodiments, the analog signal output from the conditioner unit 330 is converted by an analog to digital converter prior to being received by the controller 346. To adapt the sensor signal to a signal standard, the controller 346 may process the sensor signal further before it is sent to the physiology monitor 206. For instance a multiplying digital-analog conversion (DAC) may be performed by the controller 346 to supply digital data representing the signal measured by the sensor element and the reference voltage to the monitor 206. Additionally, other conditioning and processing may be performed by the controller 346, including the calculation of pressures, calibration, temperature compensation, and other operations as disclosed herein.

The interface unit 204 further includes the external pressure compensation module 322 that includes an external pressure sensor 360 located externally on the interface unit 204 to measure the pressure outside the patient's body and generate a signal representing the measured external pressure. The external pressure values are supplied to a pressure compensation circuit 362 which is adapted to generate a compensation value reflecting the external pressure variation during a measurement procedure. The compensation value is read by the controller 346 which may compensate the output pressure values from the sensors 140 and 142 by the compensation value prior to the output pressure signal being sent to the external physiology monitor 306.

The pressure compensation circuit 362 may include a controller and an internal memory device, (not shown in FIG. 3) When a measurement procedure is initiated to measure the pressure using the sensor circuits 310 and 312, simultaneously, or near in time to this measurement, an initial external pressure value is determined, and the compensation circuit 362 is adapted to generate real time compensation values that can be applied to subsequent pressure measurements from the sensor circuits 310 and 312 based on the difference between the present value of the external pressure detected and the initial external pressure value. Thus, each time a measurement is performed, i.e. for each pressure value obtained by the pressure sensors 140 and 142 on the guide wire 110 and generated by the sensor wire assembly 202 during a measurement procedure, the pressure value is compensated for any variation of the external pressure by adding or subtracting the pressure value determined from the guide wire sensors by the compensation value obtained from the pressure compensation circuit 362. In some embodiments, the pressure compensation circuit may calculate a direct compensation value from the external pressure at any time that may be applied to correct the pressure readings from the aorta and the left ventricle. This value may not be dependent on the history of external pressure values, and therefore may not require subtracting an initial pressure value. Therefore, a known or tested correlation between an external pressure value and a correction factor that is based on testing of the individual sensor circuits 310 and 312 (perhaps included in the calibration circuit) or average correction values, may be utilized to calculate a compensation value that can correct a pressure reading from the guide wire without comparison to external pressures taken in the beginning of the procedure. Additionally, the external pressure value taken may be compared to the pressure value used to calibrate each of the individual sensor circuits 310 and 312, and deviation from that pressure may be utilized to appropriately correct the pressure readings of the aorta and left ventricle.

The guide wire 110 is insertable into a socket or other connection of the interface unit 204. In some embodiments, the connection includes a socket has electrical contacts on its inner surface to be connected with electrode surfaces at the proximal end 122 of guide wire 110 when inserted in the socket to receive the pressure signals from the sensor circuits 310 and 312. The external pressure sensor 360 is preferably located on the interface unit 204 near the connector, but may alternatively be arranged along a connecting cable with the monitor 206 or taken by the monitor 206 itself. The interface unit 204 may also include a fastening device to hold the guide wire 110 when correctly inserted into the socket. In this example, the guide wire 110 has an outer diameter of 0.038″ and thus, the inner diameter of the socket is slightly larger than 0.038″ mm.

In some embodiments, after the controller 346 processes the received pressure signals and compensates the pressure signals with the external pressure values, the controller 346 outputs a digital data to the output interface 324 representing pressure values. The output interface 324 sends the data packets representing pressure values to the monitor 206 which may process the data packets or digital signals and display indications of the pressure values in real-time. As described above, this may include a numeric value of the pressure, a graphical representation or comparison, a color coded indication of the meaning of the pressure values, a numeric indication of the pressure difference between the aorta and left ventricle, and other values. The simultaneous detection and subsequent display of pressure readings from the aorta and the left ventricle (including the difference and other indications) does not require additionally removing a catheter 150, inserting another guide wire, or any other additional steps.

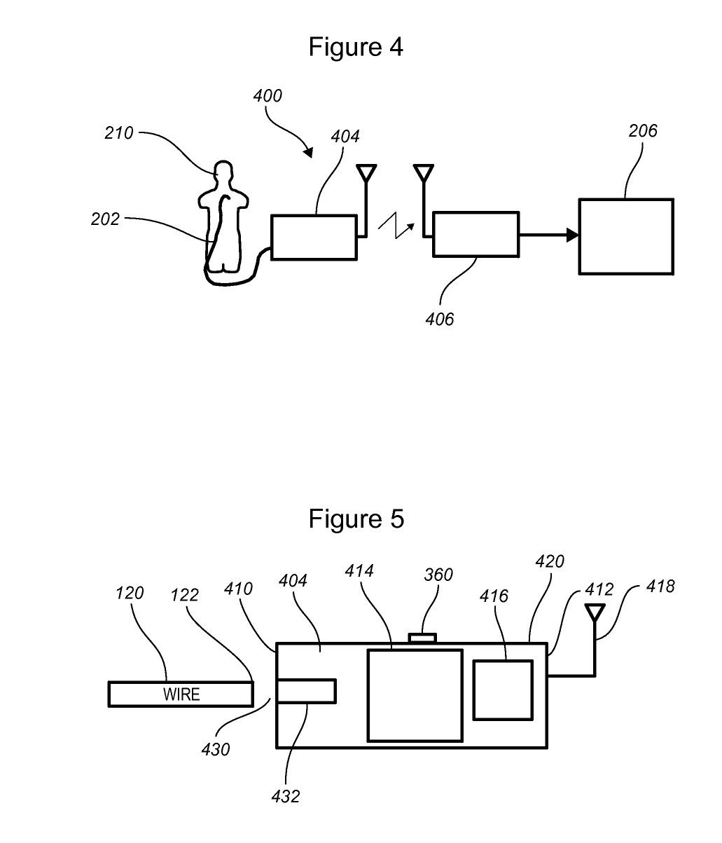

FIG. 4 is a block diagram of an alternative sensor system 400 to measure pressure from the heart and aorta using the wire assembly 202 in FIG. 1A employing wireless communication. Identical elements in FIG. 4 as those in FIG. 2 are labeled with identical element numbers. The alternative sensor system 400 includes a wireless interface unit 402 that processes the signals from the pressure sensor wire assembly 202 and wirelessly transmits the signals to a communication unit 404. The communication unit 404 is coupled to the external physiology monitor 206.

The guide wire 110 of the pressure sensor wire assembly 202 is connected, at its proximal end 122, to the wireless interface unit 404. The interface unit 404 functions similarly to the interface unit 204 in FIG. 2. The interface unit 404 includes a transceiver that is adapted to wirelessly communicate via a communication signal with the communication unit 406, and the communication unit is in turn connected to the external physiology monitor 206, in order to transfer the output pressure signal to the external physiology monitor 206. The wireless communication allows greater flexibility since the external physiology monitor 206 does not have to be in close physical proximity to the patient 210. Several other configurations could be utilized, including a wireless transmitter directly on the pressure sensors on the guide wire 110, that would transmit the data to a physiological monitor 206 over a wireless link, for example by using Bluetooth technology. In this embodiment, the signal processing and conditioning would primarily be performed on the physiological monitor 206.

FIG. 5 is a cross-section view of the interface unit 404 for the sensor system 400 in FIG. 4. The interface unit 402 has a general cylindrical shape with a connector side 410 and an opposite side 412. The interface unit 404 includes a signal processing board 414 that includes the sensor signal adaption module 320, pressure compensation module 322 and output interface 324 described in FIG. 3. In some embodiments, the interface unit 404 also has an outer surface 420 that holds the external sensor 360. The output interface 324 outputs the processed signals representing the measured pressures from the aorta and left ventricle to a transceiver unit 416. The transceiver unit 416 transmits the signals via an antenna 418 which is attached to the opposite side 412 of the interface unit 402.

In some embodiments, the connector side 410 includes an aperture 430 which leads to a cylindrical socket 432. As explained above, the proximal end 122 of the guide wire 110 is inserted through the aperture 430 into the cylindrical socket 432. The cylindrical socket 432 has electrical contacts on its inner surface (not shown) to be connected with electrode surfaces (not shown) at the proximal end 122 of guide wire 110 when inserted in the socket 432 to receive the pressure signals from the sensor circuits 310 and 312.

The wireless communication may performed by using an established communication protocol, e.g., BLUETOOTH®. Although the interface unit 404 and the communication unit 406 are described in connection with the use of a radio frequency signal it should be appreciated that different communication protocols and signals types would be equally applicable in case any alternative communication signals are used, e.g. optical or magnetic signals.

In an embodiment the TAVI pressure wire has a single left ventricular pressure sensor whose pressure waveform is compared to a waveform from a catheter in the proximal ascending aorta in lieu of the aortic pressure sensor. In some embodiments, the catheter in the proximal ascending aorta is connected to an external pressure sensor.

The pressure wire described herein may also be used in interventions other than aortic interventions. When applied to transcatheter valve interventions (TVI) other than aortic valve interventions the wire is referred to as the TVI pressure wire.

In an embodiment the TVI pressure wire has a single distal pressure sensor positioned on one side of the valve of interest whose pressure waveform is compared to a waveform from a catheter positioned on the other side of the valve of interest in lieu of a proximal pressure sensor. In some embodiments, the catheter positioned on the other side of the valve of interest is connected to an external pressure sensor.

In an embodiment the TAVI pressure wire or the TVI pressure wire have multiple pressure transducers enabling pressure monitoring at multiple points.

In an embodiment, the TAVI pressure wire not only evaluates the hemodynamics of regurgitation but also the systolic gradient across the aortic valve, thereby evaluating the hemodynamics of stenosis and immediately following or close in temporal proximity transcatheter aortic valve interventions.

In another embodiment the TVI pressure wire is inserted across the mitral valve. This is performed through a catheter inserted in the left atrium antegradely using a transseptal puncture via the femoral vein/the jugular vein/the subclavian vein, then the right atrium, through the interatrial septum to the left atrium. The wire is then advanced from the left atrium through the mitral valve, into the left ventricle. In a further embodiment the TVI pressure wire is inserted across the mitral valve retrogradely via aortic valve/apex, left ventricle, through mitral valve, then left atrium. The hemodynamics of mitral regurgitation are evaluated by comparing simultaneous left atrial and left ventricular pressure waveforms in systole. The hemodynamics of mitral stenosis are evaluated by comparing simultaneous left atrial and left ventricular pressure waveforms in diastole. The hemodynamics may be adjusted to heart rate based on data from planned research studies. The output may be used to immediately guide transcatheter mitral valve interventions.

In another embodiment the TVI pressure wire is inserted across the tricuspid valve antegradely via the femoral vein/the jugular/the subclavian vein, then the right atrium, across the tricuspid valve, to the right ventricle. The hemodynamics of tricuspid regurgitation are evaluated by comparing simultaneous right atrial and right ventricular pressure waveforms in systole. The hemodynamics of tricuspid stenosis are evaluated by comparing simultaneous right atrial and right ventricular pressure waveforms in diastole. The hemodynamics will be adjusted to heart rate based on data from planned research studies. The output will be used to immediately guide transcatheter tricuspid valve interventions.

In another embodiment the TVI pressure wire is inserted across the pulmonic valve antegradely via the femoral vein/the jugular vein/the subclavian vein then the right atrium, across the tricuspid valve, to the right ventricle, across the pulmonic valve to the pulmonary artery. The hemodynamics of pulmonic regurgitation are evaluated by comparing simultaneous right ventricular and pulmonary artery waveforms in diastole. The hemodynamics of pulmonic stenosis are evaluated by comparing simultaneous right ventricular and pulmonary artery waveforms in diastole. The hemodynamics will be adjusted to heart rate based on data from planned research studies. The output will be used to immediately guide transcatheter pulmonic valve interventions.

Paravalvular (PV) aortic regurgitation (AR) remains difficult to quantify and the utility of the AR index (ARi) to create a composite aortic insufficiency (CAI) score has been proposed. However, heart rate (HR) influences the ARi and the clinical relevance of this phenomenon remains poorly appreciated. The inventors sought to investigate the incremental prognostic value of a new composite heart rate-adjusted hemodynamic-echocardiographic aortic insufficiency (CHAI) score to the prognostic evaluation of paravalvular (PV) aortic regurgitation (AR) after balloon-expandable transcatheter aortic valve implantation (TAVI). PV AR prognostication using TEE remains challenging and is enhanced by the integration of transcatheter hemodynamics. HR-adjustment using the CHAI score provides incremental discriminatory value.

Provided herein is a method that includes providing a subject that is undergoing or has undergone trans-catheter aortic valve implantation, obtaining a transesophageal echocardiogram (TEE) to determine aortic regurgitation and determining heart rate adjusted diastolic delta in the subject. The aortic regurgitation may be classified as low, intermediate (mild or moderate) or severe based on the results of the TEE. In an embodiment, the heart rate adjusted diastolic delta is normalized to the subject's heart rate. In some embodiments, the heart rate adjusted diastolic delta of less than or equal to the reference value is indicative of poor prognosis in the subject. In some embodiments, the heart rate adjusted diastolic delta of greater than the reference value is indicative of good prognosis in the subject. In an embodiment, subject in which the heart rate adjusted diastolic delta is assessed exhibits intermediate (mild or moderate) aortic regurgitation as determined by TEE.

Further provided is a method for assessing a paravalvular leak in a subject in need thereof. The method includes providing a subject that is undergoing or has undergone transcatheter aortic valve implantation, obtaining a transesophageal echocardiogram to determine aortic regurgitation, wherein aortic regurgitation is low, intermediate (mild or moderate) or severe and determining heart rate adjusted diastolic delta in the subject with intermediate degrees (mild or moderate) of aortic regurgitation. In an embodiment, the heart rate adjusted diastolic delta is normalized to the subject's heart rate. In some embodiments, the heart rate adjusted diastolic delta of less than or equal to the reference value is indicative of a intermediate (mild or moderate) to severe paravalvular leak in the subject. In some embodiments, the heart rate adjusted diastolic delta of greater than the reference value is indicative of a no paravalvular leak or a mild paravalvular leak in the subject. In some embodiments, if the heart rate is considered acceptable, analysis of pressure waveforms recorded by the TAVI/TVI pressure wire will incorporate other algorithms that do not adjust for heart rate and compare the different pressure waveforms by alternative algorithms and/or formulae.

Also described herein is a method for treating a paravalvular leak in a subject in need thereof. The method includes providing a subject that is undergoing or has undergone transcatheter aortic valve implantation, obtaining a transesophageal echocardiogram to determine aortic regurgitation, wherein aortic regurgitation is low, intermediate (mild or moderate) or severe and determining heart rate adjusted diastolic delta in the subject with moderate aortic regurgitation and prescribing a therapy to the subject if the subject has poor prognosis, so as to treat the paravalvular leak in the subject. In an embodiment, the heart rate adjusted diastolic delta is normalized to the subject's heart rate. In some embodiments, the heart rate adjusted diastolic delta of less than or equal to the reference value is indicative of poor prognosis in the subject and the heart rate adjusted diastolic delta of greater than the reference value is indicative of good prognosis in the subject.

In various embodiments, treatments for TAVI aortic paravalvular regurgitation include but are not limited to balloon post-dilatation.

The reference value is obtained from an ongoing database of outcomes in patients undergoing TAVI and is defined as the cut-off below which there is a strong correlation with adverse clinical outcome. This may be determined by several statistical methods including but not limited to ROC curve analyses and case-control studies.

The heart rate adjusted diastolic delta is calculated according to the Formula 1:

HRA-DD=(AoDBP−LVEDP)/HR (2)

wherein the AoDBP is the aortic diastolic blood pressure, LVEDP is the left ventricular end-diastolic pressure and HR is the heart rate. A HRA-DD less than the reference value is indicative of significant paravalvular AR. The heart rate may be derived from either the electrocardiogram externally or from the frequency of pressure pulsations derived from the pressure wire sensor (for example the guide wire that includes one or two pressure sensors for use in TAVI as described herein).

In some embodiments, the HRA-DD may be multiplied by a constant (X) so as to generate a simplified number that is easier for physicians to remember (for example, see Example 4 herein). In such an instance, the heart rate adjusted diastolic delta is calculated according to Formula 3:

HRA-DD=(AoDBP−LVEDP)/HR*X (3)

For instance, if HRA-DD is multiplied by 80 (i.e. X=80), a reference value of 25 was found in a single center series by the inventors to be clinically significant such that intermediate AR by TEE with an HRA-DD<25 best stratified survival outcomes.

In some embodiments, the blood pressure for determining the CHAI score is obtained using the guide wire comprising two pressure sensors as described herein. In some embodiments, the blood pressure for determining the CHAI score may be obtained using any device that provides the aortic diastolic blood pressure and the left ventricular end-diastolic blood pressure.

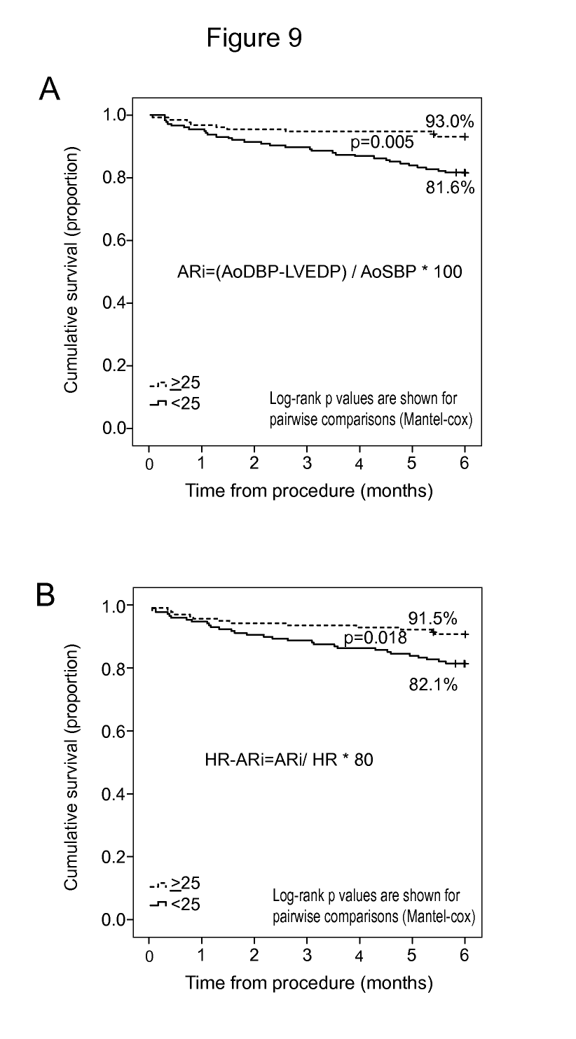

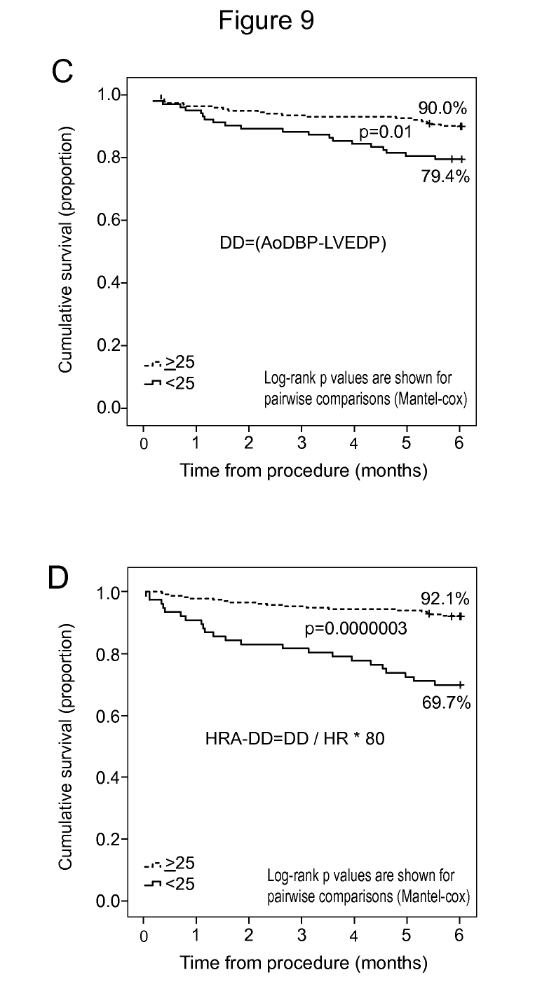

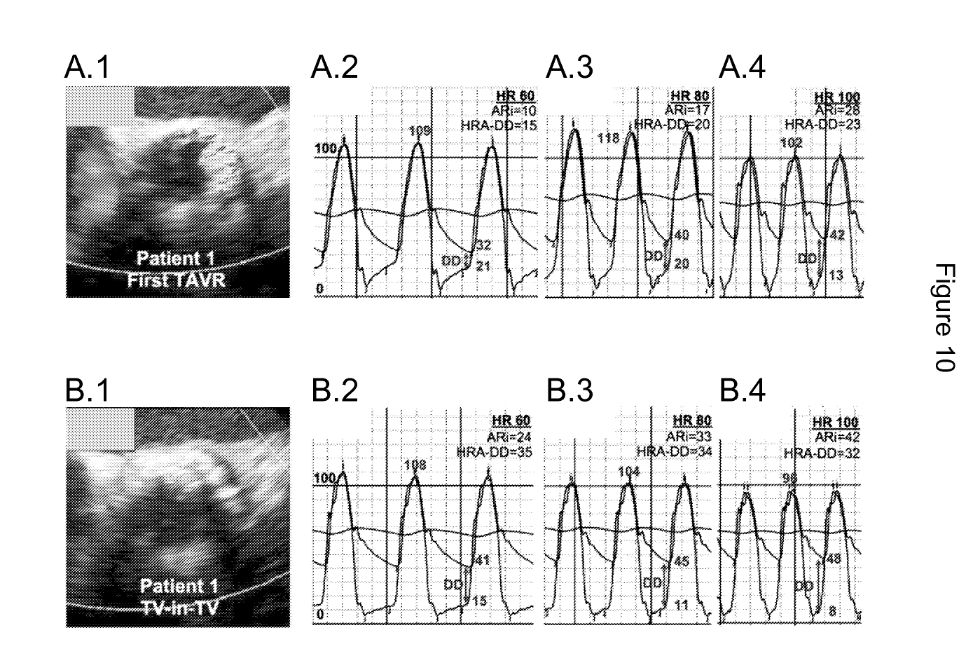

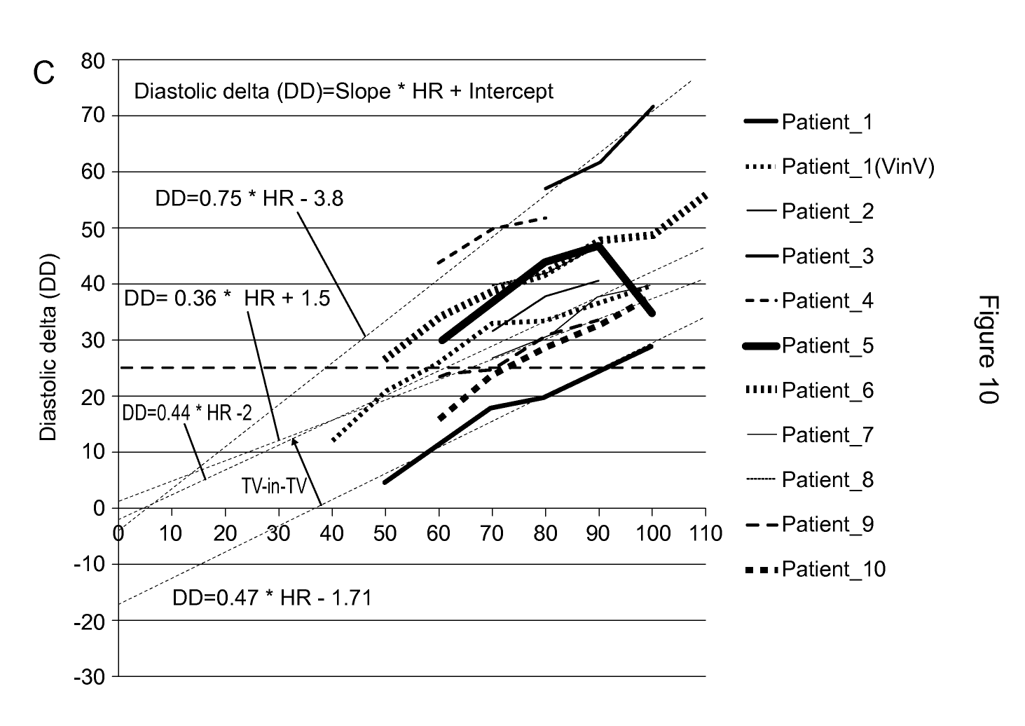

The CHAI score described herein is advantageous because it improves substantially on the ARi as the ARi may be falsely low or in a low heart rate (FIG. 10B.2) and falsely high if the heart rate is high (FIG. 10A.4). This may lead to incorrect conclusions and potentially inappropriate over or under-treatment unless the variability in heart rate is compensated for. Importantly, unlike HR adjustment of the DD which substantially improved survival stratification, HR adjustment of the ARi did not improve on the prognostic value of the ARi (FIG. 9A-D).

In some embodiments analysis of pressure waveforms recorded by the TAVI/TVI pressure wire will incorporate other algorithms that adjust for heart rate, such as the diastolic:systolic velocity time integral ratio, or alternative algorithms and/or formulae adjusting for heart rate.

### EXAMPLES

The inventors (i) evaluated the prognostication of PV AR by Valve Academic Research Consortium 2 (VARC 2) derived TEE grading alone⁹, (ii) objectively sought hemodynamic parameters other than the ARi, incorporating heart rate adjustment, that might better predict outcome, (iii) used this evidence-based data to generate an optimal composite heart-rate adjusted hemodynamic-echocardiographic aortic insufficiency (CHAI) score, (iv) tested its incremental value for the prognostication of PV AR after TAVR of this CHAI score in the context of VARC2 TEE criteria and a composite hemodynamic-echocardiographic aortic insufficiency without heart rate adjustment (CAI) score, in line with a recently proposed methodology¹⁰ and lastly (v) examined the baseline associations of this CHAI score and its ability to predict outcome in a multivariable model for mortality.

### Example 1

### Experimental Methods

### Patient Population, Assessment and Procedure

All patients had severe symptomatic aortic stenosis (AS) and were treated in a single center with balloon-expandable TAVR (Edwards Sapien/Sapien XT, Edwards Lifesciences LLC.), performed under predominant fluoroscopic guidance, as has been previously described¹¹. All patients studied had simultaneous transcatheter transaortic hemodynamic pressures measured post TAVR, with a multipurpose catheter placed across the transcatheter valve into the left ventricular cavity and a pigtail catheter placed in the aortic root above the transcatheter valve. If an additional maneuver was performed, such as valve-in-valve or post-dilatation, hemodynamic pressures were recorded after that additional intervention.

Patients also had peri-procedural TEE imaging for procedural guidance and post TAVR evaluation of valvular function. TEE was performed using the iE33 xmatrix echocardiography system (Philips Ultrasound, Philips Medical Systems, Bothell, Wash.). Within the confines of available transcatheter hemodynamic data, patients were consecutive and all were followed beyond 1-year after the index procedure (all patients had at least 1-year post-procedural follow-up).

Sizing for TAVR was made at the operator's discretion, using data from all available imaging modalities at the time of the procedure, with a reliance on traditional cut-offs for annular size by 2D-TEE measurement (D2D-TEE) early in the series, and a later reliance predominantly on cross-sectional measurements by computed tomography or three-dimensional echocardiography^{12, 13}.

### Post TAVR PV AR, the ARi and Heart Rate

Post TAVR PV AR was assessed in line VARC-2 criteria¹⁴, with peri-procedural TEE examinations reviewed retrospectively. This was performed by one of 2 physician readers experienced in the assessment of TAVR echocardiograms, blinded to the peri-procedural TEE report, annular measurements, clinical, angiographic and hemodynamic data. Reproducibility was excellent: for intra-observer agreement for the assessment of significant PV regurgitation, the kappa statistic was 0.77 (p<0.001), and for inter-observer agreement, the kappa was also 0.77 (p<0.001)⁵. The transcatheter ARi index was calculated according to the following formula: [(DBP-LVEDP)/SBP]×1006. An ARi<25 was regarded as clinically significant⁶. The heart rate (HR) was derived from the simultaneous electrocardiogram using the R-R interval associated with the hemodynamic waveform studied with stable electrocardiogram and hemodynamics for at least 3 beats. This was used to generate the heart-rate adjusted diastolic delta (HR-DD), calculated as [DD/HR], where diastolic delta was (aortic diastolic pressure minus left ventricular end-diastolic pressure).

### Statistical Analysis

Statistical analyses were made using SPSS software (PASW v18, SPSS Inc, Chicago, Ill.) and MedCalc v12.7.0 (MedCalc, Ostend, Belgium). Normality of distributions for continuous variables was tested using the Shapiro-Wilks test and data analyzed appropriately thereafter.

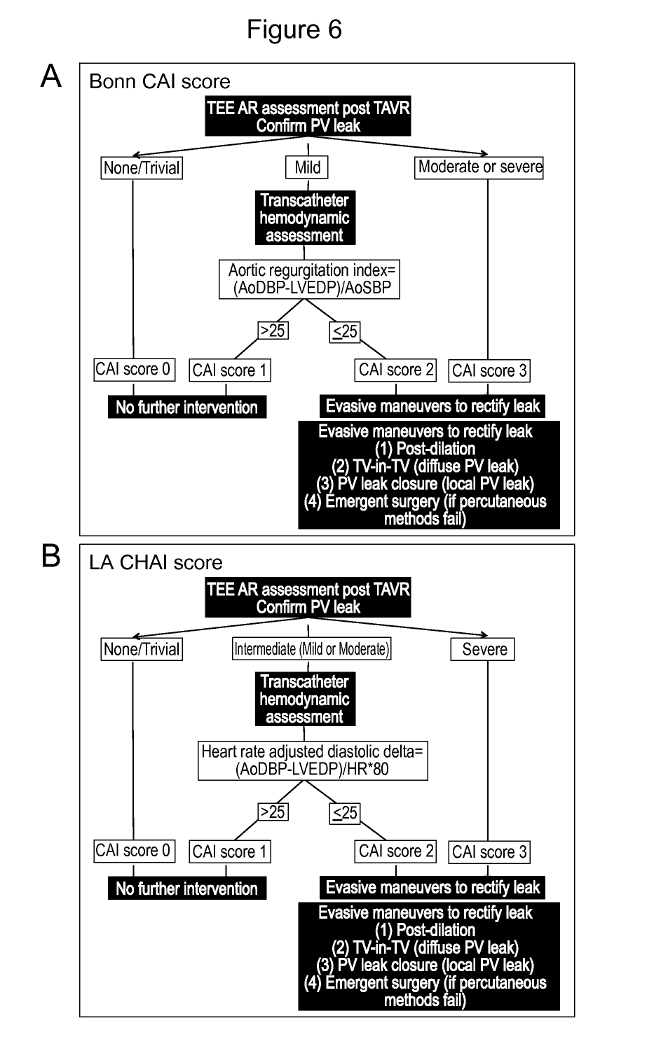

Other hemodynamic parameters were also studied for their predictive value for 1-year mortality using Receiver Operator Characteristic (ROC) curve analysis. The hemodynamic parameter most predictive of survival was combined with TEE AR grade data to generate an optimal composite (TEE/hemodynamic) heart rate adjusted AI (CHAI) score. This was based on the TEE AR grade if there was none/trivial (graded CHAI 0) or severe AR (graded CHAI 3) on TEE or on a combination of TEE and heart-rate adjusted transcatheter hemodynamics if there was intermediate AR (mild or moderate) (FIG. 6B). Intermediate PV AR was graded as not significant if the HR-DD was ≧reference value (CHAI score 1) and significant if the HR-DD was <reference value (CHAI score 2). A composite AI (CAI) score, recently proposed by the Bonn group, incorporating the ARi without heart rate adjustment has suggested that AR≧moderate by angiography or echocardiography be regarded as significant and the ARi (<25) be used to stratify mild AR for significance FIG. 6A.

ROC curves were generated using post TAVR 1-year mortality as the end-point (state variable) and VARC-2 TEE AR grade, CAI score and CHAI score as the studied variables. The method of deLong et al¹⁵ was used for direct comparisons of the discriminatory value of one modality to another. Kaplan-Meier curves were also studied for 1-year survival stratified according to these respective groups.

A multivariable model for 1-year mortality incorporating baseline and peri-procedural variables associated with 1-year mortality to a significance ≦0.1 was employed using a forward: LR analysis. This included age, male sex, baseline creatinine >2 mg/dl, pulmonary disease, STS score, baseline peak velocity, heart rate and LV ejection fraction. In order to further establish the dominant prognostic modality assessing PV AR, the three competing parameters PV AR≧moderate by TEE, CAI score≧2 and CHAI score≧2 were progressively added to the model.

### Statistical Methods

The Fisher exact test was used for categorical variables compared across independent groups. For normally distributed continuous variables compared across independent groups, an independent samples t-test was employed. For non-normally distributed continuous variables compared across independent groups, a Mann Whitney U test was used.

### Example 2

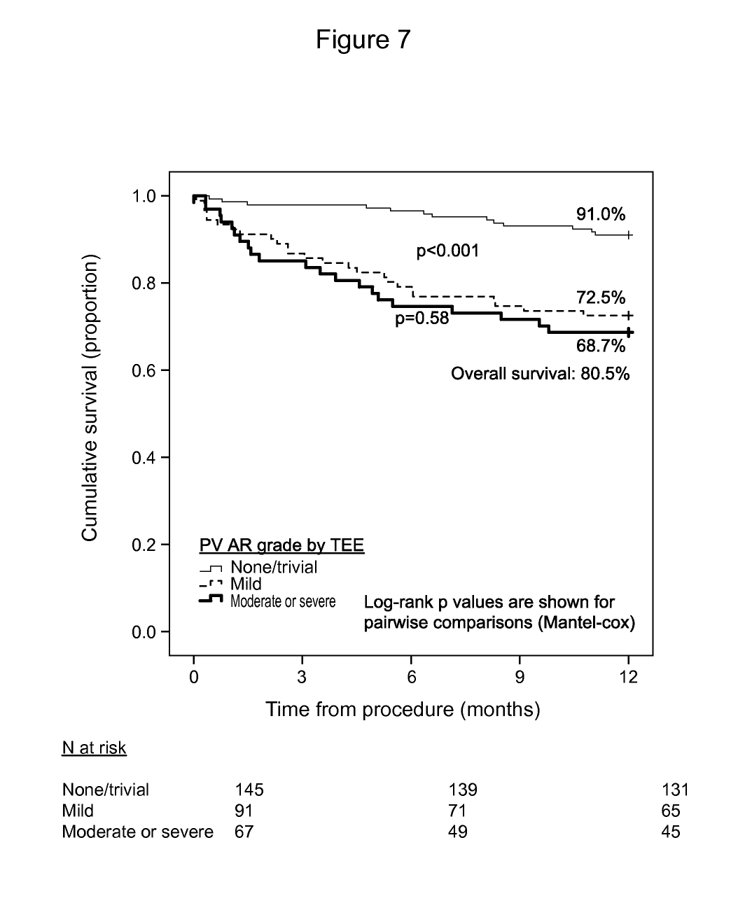

A total of 303 patients were studied. Median age was 86 (interquartile range, IQR, 80-90) and mean aortic valve gradient was 43 mmHg (IQR 41-52). By TEE VARC-2 criteria, 145 had no/trivial PV AR (47.9%), 91 had mild PV AR (30.0%), 62 had moderate (20.5%) and 5 severe PV AR (1.7%). Overall, PV AR by TEE stratified survival poorly (FIG. 7). Although there was an excellent prognosis if there was no or trivial PV AR by TEE, there was considerable overlap in outcomes amongst patients in the intermediate range of echocardiographic severity with mild and moderate/severe PV AR having similarly poor outcomes (FIG. 7).

### Example 3

### Paravalvular Regurgitation, the Aortic Regurgitation Index (ARi) and Heart Rate

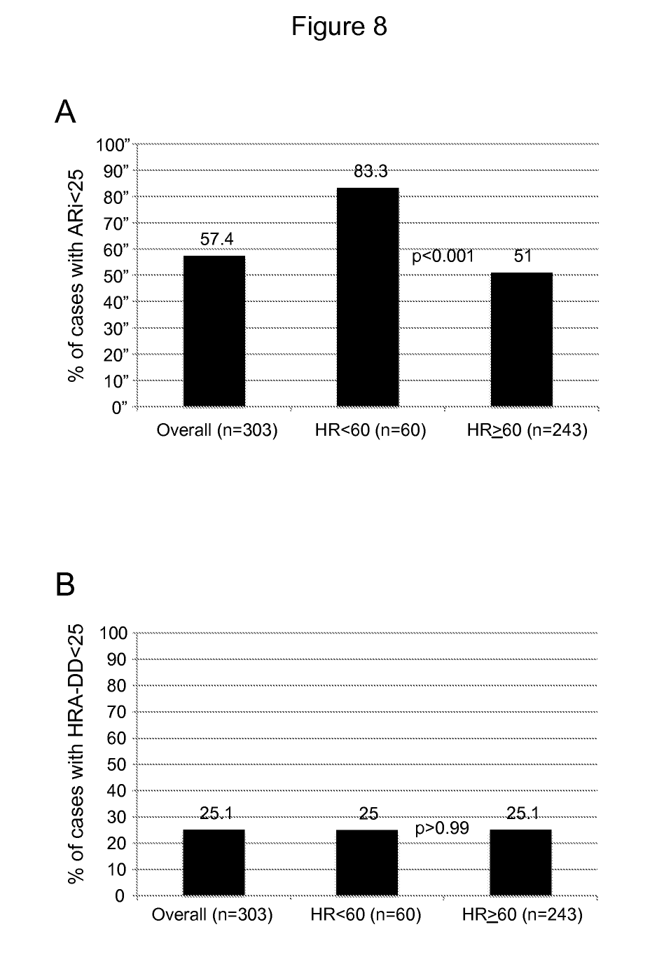

A total of 60 patients (19.8%) had a HR<60, 187 (61.7%) a HR 60-80 and 56 (18.5%) a HR>80. HR was unrelated to PV AR grade by TEE (r=0.04, p=0.48). ARi was weakly correlated to both TEE PV AR grade (r=−0.20, p=0.001) and heart rate (r=0.30, p<0.0001). In a bivariate binary regression model for ARi<25, both HR<60 (OR 5.2, 95% CI 2.5-10.7, p<0.0001) and PV AR grade≧2 (OR 2.0, 95% CI 1.1-3.5, p=0.024) were significant determinants of ARi<25. The higher OR and lower p value of HR<60 suggested a greater contribution of bradycardia to a low ARi than higher PV AR grade. Indeed, despite no relationship between HR and PV AR by TEE, 50/60 (83.3%) of those with HR<60 had an ARi<25 compared to only 19/56 (33.9%) with a HR>80 (FIG. 8A-B).

### Example 4

### Heart Rate Adjustment of the ARi (HRA-ARi)

In an attempt to correct for the influence of HR on the ARi, a simple heart rate adjustment of the ARi was performed by the formula [HR adjusted ARi (HRA-ARi)=ARi/HR*80]. A HR of 80 was selected since at this HR the highest sum of sensitivity and specificity occurred at a HRA-ARi≦24, preserving the previously suggested cut-off of ARi<25; adjustment to a HR of 72 resulted in the highest sum of sensitivity and specificity occurring at a HRA-ARi≦21. Simple adjustment of HR using the HRA-ARi did not seem to improve the stratification of 1-year survival (FIG. 9A-D).

### Example 5

### Optimal Diastolic Hemodynamic Indices for Prognostication

We further studied transcatheter hemodynamic parameters related to survival. A comparison of the individual components of the ARi (AoDBP, LVEDP and AoSBP), showed the “diastolic delta” (DD, the difference between aortic diastolic and LV end diastolic pressure) to have the greatest predictive value for 1-year mortality (table 1). This improved further with simple heart rate adjustment (Diastolic delta/HR*80). Simple heart rate adjustment of the DD dramatically improved the stratification of 1-year survival (FIG. 9A-D). The heart rate adjusted diastolic delta (HRA-DD) removed the substantial influence of bradycardia on transcatheter hemodynamics (FIG. 8B) and was therefore the preferred hemodynamic measure and was employed for the remainder of the study. The highest sum of sensitivity and specificity for 1-year mortality by the HRA-DD occurred at a threshold of ≦24.8. The cut-off of 25 was therefore retained for simplicity. Of note, although the AoSBP is the denominator of the ARi (and hence a lower AoSBP would increase the ARi), lower AoSBP was associated with higher 1-year mortality (Table 1).

TABLE 1
Receiver operator characteristic analyses of hemodynamic
parameters with 1-year mortality as the end-point.

| | | 95% Confidence | | |
|---|---|---|---|---|
| | | Interval | | |
| All patients (n = 303) | Area | Lower | Upper | P |
| Post TAVR AoDBP | 0.59 | 0.50 | 0.67 | 0.035 |
| -LVEDP | 0.58 | 0.49 | 0.66 | 0.076 |
| Post TAVR AoSBP | 0.61 | 0.53 | 0.69 | 0.007 |
| ARi | 0.60 | 0.52 | 0.68 | 0.019 |
| HRA-ARi (HR 80) | 0.63 | 0.55 | 0.71 | 0.001 |
| DD | 0.64 | 0.56 | 0.72 | 0.001 |
| HRA-DD (HR 80) | 0.68 | 0.60 | 0.76 | <0.001 |

TAVR—transcatheter aortic valve replacement;
AoDBP—aortic diastolic blood pressure;
LVEDP—left ventricular end diastolic blood pressure;
ARi—aortic regurgitation index;
AoSBP—aortic systolic blood pressure;
HRA—heart rate adjusted;
HR—heart rate;
DD—diastolic delta.

### Example 6

### The Effect of Ventricular Pacing on the ARi and Diastolic Hemodynamics

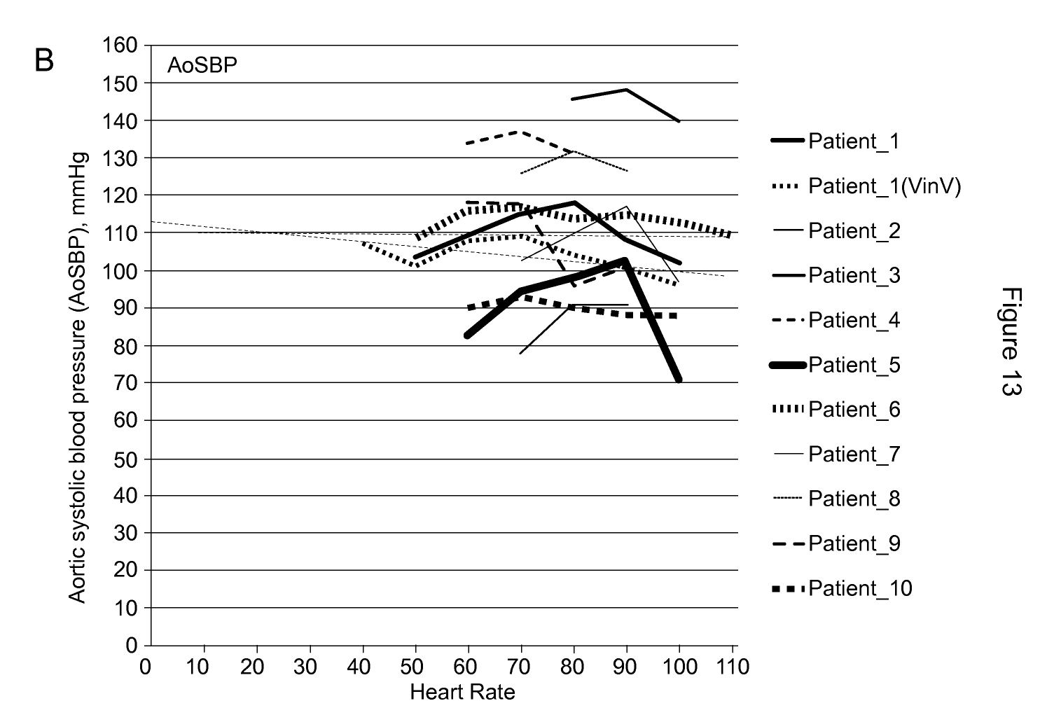

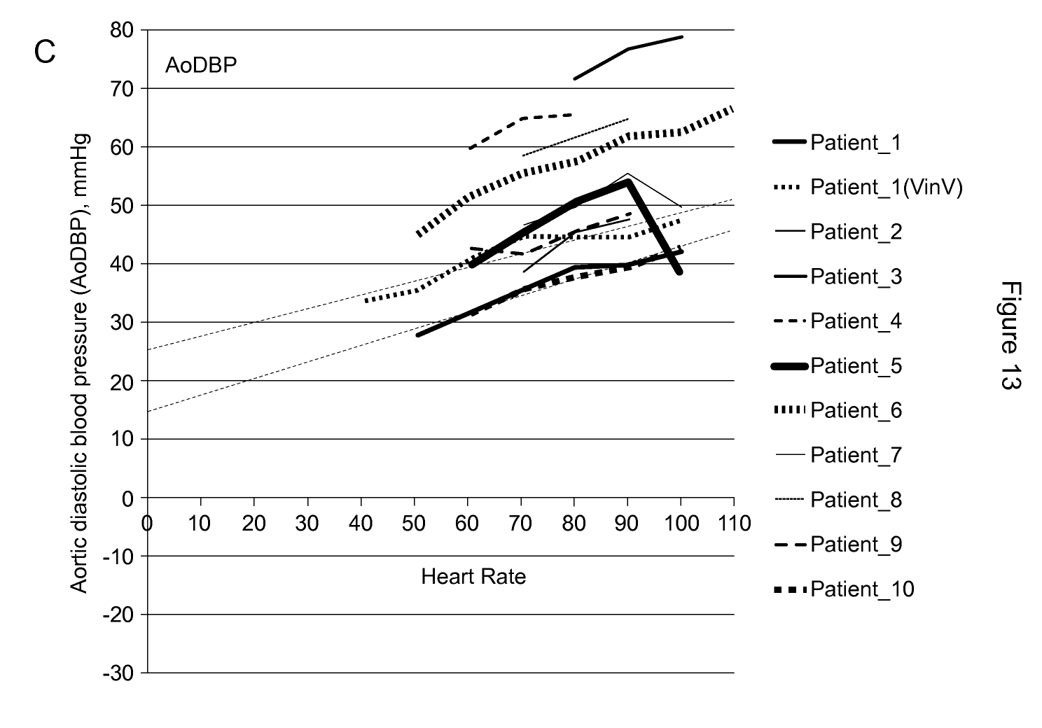

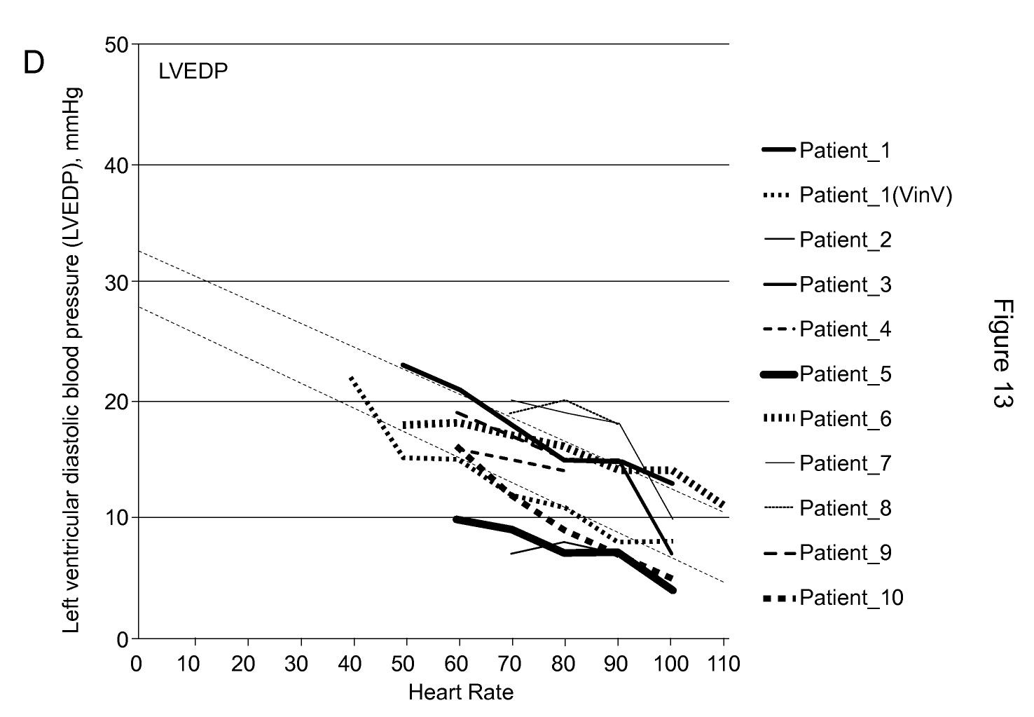

Given that simple HR adjustment could not improve the prognostic performance of the ARi in the setting of relative bradycardia, we also investigated the impact of ventricular pacing on the immediate post TAVR transcatheter hemodynamics in a subsequent cohort of patients (FIGS. 10A.1-A.4, 10B.1-B.4 and C; FIG. 13A-D). After calculating the ARi and the diastolic delta at the endogenous rate, we ventricular paced at a rate of 100 and then decrements of 10 until the endogenous rate returned. The ARi, AoDBP, diastolic delta steadily increased and LVEDP decreased in line with increasing heart rate (FIGS. 10A.1-A.4, 10B.1-B.4 and C; FIG. 13A-D). The denominator of ARi, the AoSBP, in contrast, displayed a flat relationship with heart rate. A mathematical model demonstrated linearity of changes in the ARi and DD with heart rate but formulae derived from this model did not add to the prognostic value of simple HR adjustment of the ARi or the DD. Simple HR-adjustment of the diastolic delta was therefore favored as a hemodynamic prognosticator, given the combination of simplicity and similarly high correlation to outcome.

### Example 7

### A Composite Hemodynamic-Echocardiographic Aortic Insufficiency Assessment (CAI)

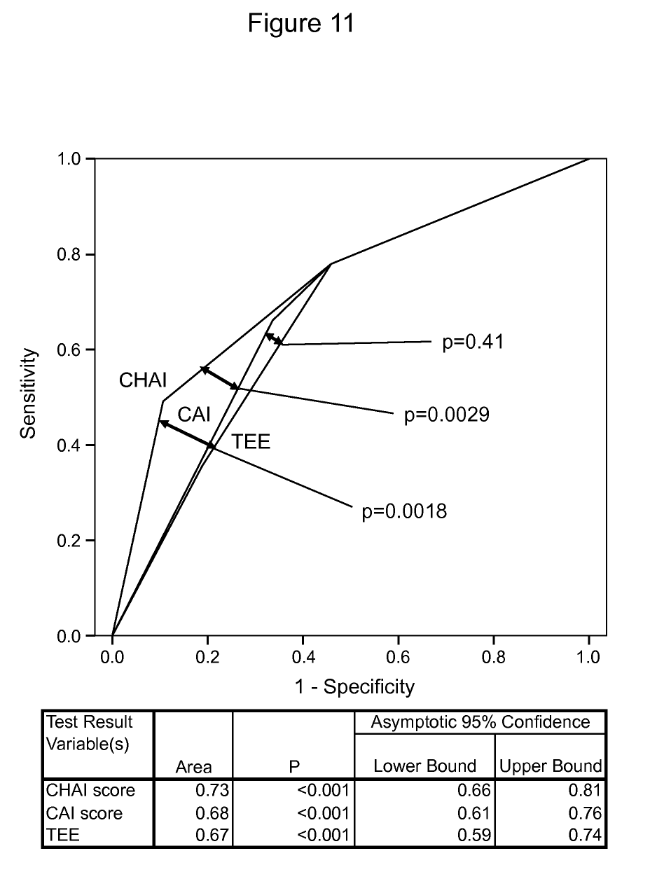

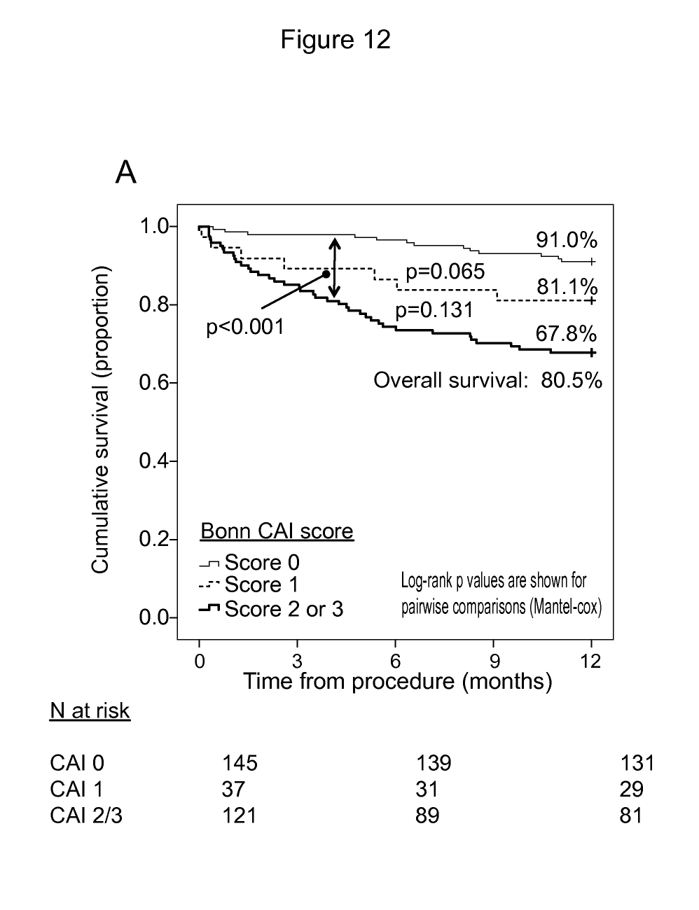

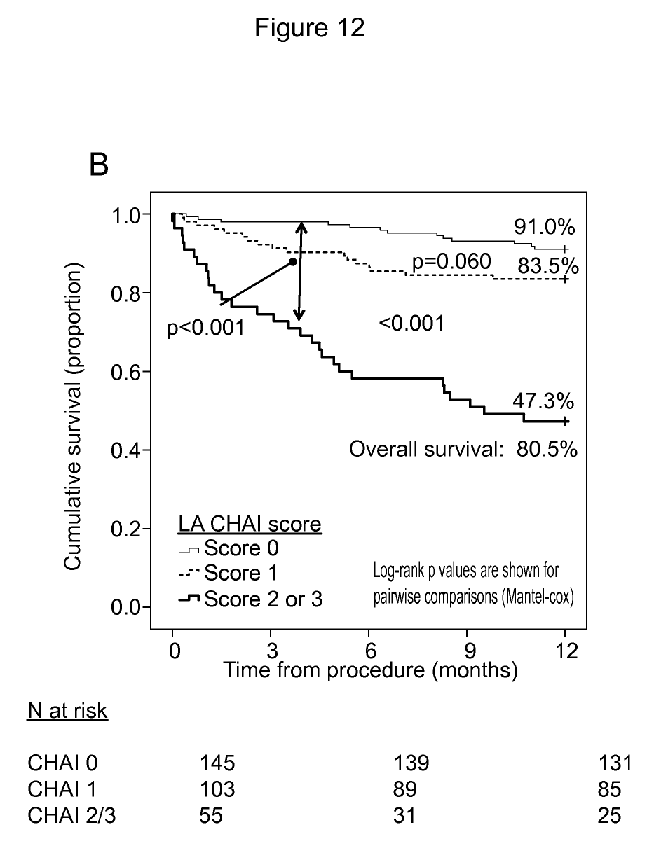

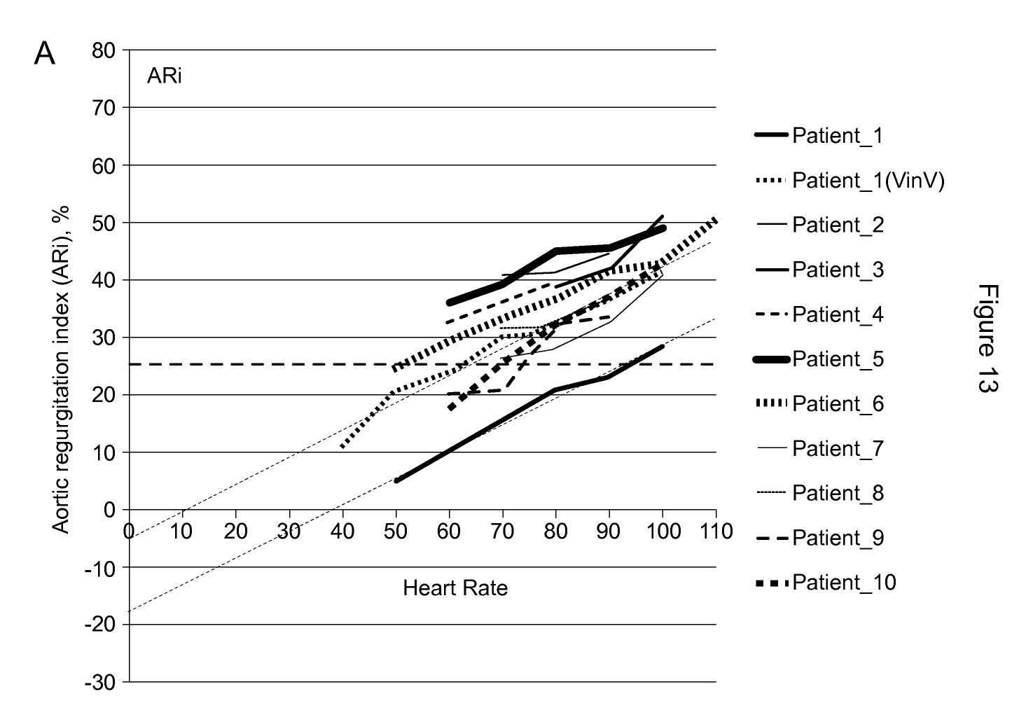

Composite hemodynamic-echocardiographic assessment using the CAI score, in line with the methodology proposed by the Bonn group¹⁰, stratified survival somewhat better than TEE alone (FIG. 11). However, the hemodynamically non-significant CAI score patients still had a prognosis that was clearly disparate to the group with no/trivial AI and intermediate between the former and the significant CAI score group.

### Example 8

### Incremental Prognostic Value of the CHAI Score: Survival

Since the extremes of PV AR by TEE stratified survival well, transcatheter hemodynamics were not applied for these patients, who retained their TEE grade separation in the composite TEE-hemodynamic grading (0 for none/trivial and 3 for TEE graded AR≧3). Given the difficulty in assessing “intermediate” (mild or moderate) and the superimposed outcomes seen in this range (FIG. 7), the simple heart-rate adjustment of the diastolic delta (Diastolic delta/HR*80) was applied to these patients and those with a value ≧25 were graded 1 in the CHAI score and those with a value <25 were graded 2.

The CHAI score was compared to the CAI score and TEE alone for discrimination of 1-year mortality using ROC curve analysis (FIG. 11): the composite assessment without heart rate adjustment (Bonn CAI score) was not superior to TEE (Bonn CAI score AUC 0.69, 95% CI 0.63 to 0.74 vs TEE AUC 0.67, 95% CI 0.62 to 0.72, p for difference 0.30). In contrast, the composite assessment with heart rate adjustment (Cedars CHAI score) was superior to both TEE (Cedars CHAI score AUC 0.73, 95% CI 0.68 to 0.78 vs TEE AUC 0.67, 95% CI 0.62 to 0.72, p for difference 0.002) and the Bonn CAI score (Cedars CHAI score AUC 0.732, 95% CI 0.68 to 0.78 vs Bonn CAI score AUC 0.69, 95% CI 0.63 to 0.74, p for difference 0.006).

For patients in the intermediate range of PV AR by TEE (mild or moderate, n=153), there were 91 with mild and 62 with moderate PV AR by VARC2 criteria. For each of the intermediate categories, composite hemodynamic assessment found 29.7% and 37.1% respectively to be clinically significant (ie. HRA-DD<25 giving CHAI=2). For each respective category of mild and moderate PV AR by VARC2 TEE criteria, if the CHAI score was clinically significant (CHAI=2) vs not (CHAI=1) the 1-year mortality was 48.1% vs 18.8% (p=0.009) for mild PV AR by TEE and 56.5% vs 12.8% (p<0.001) for moderate PV AR by TEE. Overall, for intermediate PV AR by TEE, the CHAI score stratified the 1-year mortality at 52.0% vs 16.5% (p<0.001).

### Example 9

### The CHAI Score, Left Ventricular Chamber Dimensions and Natriuretic Peptides

A CHAI score>1 vs. ≦1 stratified the 1 month left ventricular end-systolic dimension expressed as a percentage of baseline; this was 106% baseline (IQR 93.8-119.2) vs. 96% baseline (IQR 88-110.2) respectively (p=0.019). Neither AR≧moderate vs. <moderate by VARC2 TEE criteria (p=0.19) or CAI>1 vs ≦1 (p=0.20) stratified this parameter. Percentage change in serum natriuretic peptide (NPA) levels at 1-3 months post-procedure relative to baseline did not differ significantly in those with AR≧moderate vs. <moderate by TEE (p=0.12) or in those with CAI>1 vs ≦1 (p=0.15). In contrast, the percentage change in serum NPA levels at 1-3 months post-procedure relative to baseline was better stratified by CHAI score (for CHAI score>1 vs. ≦1 104.4% of baseline [IQR 49.5-239.1] vs. 78.5% of baseline [IQR 53.7-130.7]), although this was of borderline statistical significance p=0.051.

### Example 10

### Multivariable Analysis of 1-Year Mortality and the Optimal Prognosticator for PV AR

In univariate analysis, variables related to 1-year mortality to a p<0.1 included age, male sex, baseline creatinine>2 mg/dl, pulmonary disease, STS score, baseline peak velocity, heart rate, LV ejection fraction, PV AR≧moderate by TEE, CAI score≧2 and CHAI score≧2. In the multivariable model without CAI and CHAI scores, PV AR≧moderate by TEE was not a statistical predictor of 1-year mortality (p=0.072), whereas male sex (OR 4.11, 95% CI 1.93-8.76, p<0.0001), baseline creatinine>2 mg/dl (OR 2.78, 95% CI 1.29-5.98, p=0.009) and HR (per 10 beats-per-minute increase in HR, OR 1.22, 95% CI 1.02-1.45, p<0.030) were significant independent predictors. The CAI score was a significant independent risk factor for death when it was added to the model (OR 3.31, 95% CI 1.60-6.84, p=0.001). In turn, addition of the CHAI score to this model rendered the CAI score non-significant (p=0.12), whereas the CHAI score emerged as the dominant predictor of death at 1-year (OR 6.5, 95% CI 3.1-13.8, p<0.001) when the 3 competing variables assessing PV AR were all included in the model (table 2).

TABLE 2
Multivariable analysis of the predictors of 1-year mortality.
A forward: LR binary logistic regression model was employed

| | OR | 95% CI | | Univariate P | OR | 95% CI | | Multivariate P |
|---|---|---|---|---|---|---|---|---|
| LA CHAI score ≧2 | 8.11 | 4.22 | 15.57 | <0.001 | 6.50 | 3.06 | 13.79 | 0.001 |
| Bonn CAI score ≧2 | 3.85 | 2.11 | 7.03 | <0.001 | Dropped | | | |
| Baseline creatinine | 3.33 | 1.66 | 6.68 | 0.00 | 2.33 | 1.03 | 5.26 | 0.04 |
| over 2 mg/dl | | | | | | | | |
| Male sex | 3.10 | 1.65 | 5.80 | <0.001 | 2.40 | 1.09 | 5.27 | 0.03 |
| PV AR ≧ moderate | 2.38 | 1.28 | 4.43 | 0.01 | Dropped | | | |
| Pulmonary disease | 1.64 | 0.91 | 2.95 | 0.10 | Dropped | | | |
| Peak velocity (per | 1.60 | 1.05 | 2.44 | 0.03 | Dropped | | | |
| m/s lower velocity) | | | | | | | | |
| Age (per 10 years | 1.45 | 1.04 | 2.02 | 0.03 | Dropped | | | |
| younger) | | | | | | | | |
| Ejection fraction | 1.29 | 1.08 | 1.55 | 0.01 | Dropped | | | |
| (per 10% lower) | | | | | | | | |
| Heart rate (per 10 | | | | | | | | |
| beats/min higher | 1.18 | 1.00 | 1.39 | 0.06 | Dropped | | | |
| rate) | | | | | | | | |
| STS score | 1.06 | 0.99 | 1.14 | 0.10 | Dropped | | | |

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal; a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Bonow R O, et al. J Am Coll Cardiol. 2008; 52:e1-142.
2. Kodali S K, et al. N Engl J Med. 2012; 366:1686-1695.
3. Jilaihawi H, Makkar R R. EuroIntervention. 2012; 8:31-33.
4. Gilard M, et al. N Engl J Med. 2012; 366:1705-1715.
5. Généreux P, et al. J Am Coll Cardiol. 2013; 61:1125-1136.
6. Sinning J M, et al. J Am Coll Cardiol. 2012; 59:1134-1141.
7. Vasa-Nicotera M, et al. J Am Coll Cardiol Intv. 2012; 5:858-865.
8. Jilaihawi H, et al. Cardiology clinics. 2011; 29:211-222.
9. Kappetein A P, et al. J Am Coll Cardiol. 2012; 60:1438-1454.
10. Sinning J M, et al. J Am Coll Cardiol. 2013; 62:11-20.
11. Leon M B, et al. N Engl J Med. 2011; 363:1597-1607.
12. Jilaihawi H, et al. J Am Coll Cardiol. 2012; 59:1275-1286.
13. Jilaihawi H, et al. J Am Coll Cardiol. 2013; 61:908-916.
14. Leon M B, et al. J Am Coll Cardiol. 2011; 57:253-269.
15. DeLong E R, et al. Biometrics. 1988; 44:837-845.
16. Sherif M A, et al. EuroIntervention. 2011; 7:57-63.
17. Daneault B, et al. Circulation: Cardiovascular Interventions. 2013; 6:85-91.
18. Willson A B, et al. J Am Coll Cardiol. 2012; 59:1287-1294.
19. Nombela-Franco L, et al. J Am Coll Cardiol Intv. 2012; 5:499-512.
20. Barbanti M, et al. J Am Coll Cardiol. 2013; 61.
21. Makkar R, et al. J Am Coll Cardiol. 2013; In Press.
22. Rihal C S, et al. J Am Coll Cardiol Intv. 2012; 5:121-130.

1. A pressure sensor wire assembly for measuring pressure in the heart of a patient, the assembly comprising:
a guide wire, having an aortic section and a ventricle section, the guide wire being insertable through a heart;
an aortic pressure sensor on the aortic section of the guide wire configured to sense pressure in the aorta;
a ventricular pressure sensor on the ventricle section of the guide wire configured to sense pressure in the ventricle, wherein the distance between the aortic pressure sensor and the ventricular pressure sensor along the guide wire is configured to allow the aortic pressure sensor to be located in the aorta while the ventricle pressure sensor is simultaneously located in the left ventricle; and
an interface on a distal end of the guide wire to send signals output from the aortic and ventricular pressure sensors.

2. The assembly of claim 1, further comprising:
a sensor signal adapting circuitry, being an integrated part of the assembly, wherein the sensor signal adapting circuitry is configured to process signals generated by the aortic pressure sensors to output aortic pressure data that represents the aortic pressure and process signals generated by ventricular pressure sensor and output data representing the ventricular pressure.

3. The assembly of claim 2, further comprising:
an external pressure sensor arranged in the assembly, configured to measure an external pressure outside the patient's body, and configured to generate an external pressure value based on the measurement of the external pressure;
a pressure compensator in the assembly configured to process the external pressure value and output a compensation value based on the measured external pressure, and modify the aortic pressure data and ventricular pressure data based on the compensation value in order to compensate for external pressure variation.

4. The assembly of claim 2, wherein the interface includes a transceiver to wirelessly communicate the pressure signals to the external physiology monitor.

5. The assembly according to claim 1, wherein the interface unit has an elongated aperture adapted to receive a proximal end of the guide wire.

6. The assembly of claim 5, wherein the interface is a general cylindrical shape allowing the generation of torque when attached to the guide wire.

7. The assembly of claim 1, wherein the pressure sensors include piezoresistors arranged in a bridge and a diaphragm.

8. The assembly of claim 1, wherein the interface includes a controller to receive the signals and convert the signals into a digital format, and a calibration circuit to calibrate the sensors.

9. The pressure sensor wire assembly of claim 1, wherein the guide wire has an outer diameter of 0.035″ or 0.038″.

10. A method comprising:
(i) providing a subject that is undergoing or has undergone transcatheter aortic valve implantation;
(ii) obtaining a transesophageal echocardiogram and determining whether aortic regurgitation is mild, moderate or severe; and
(iii) determining heart rate adjusted diastolic delta if the subject has intermediate aortic regurgitation,
wherein the heart rate adjusted diastolic delta of less than or equal to the reference value is indicative of poor prognosis in the subject and the heart rate adjusted diastolic delta of greater than the reference value is indicative of good prognosis in the subject.

11. A method for assessing a paravalvular leak in a subject in need thereof comprising:
(i) providing a subject that is undergoing or has undergone transcatheter aortic valve implantation;
(ii) obtaining a transesophageal echocardiogram and determining whether aortic regurgitation is mild, moderate or severe; and
(iii) determining heart rate adjusted diastolic delta if the subject has intermediate aortic regurgitation,
wherein the heart rate adjusted diastolic delta of less than or equal to the reference value is indicative of a clinically significant paravalvular leak in the subject and the heart rate adjusted diastolic delta of greater than the reference value is indicative of a non-clinically significant paravalvular leak in the subject.

12. (canceled)

13. The method of claim 10 or 11, wherein the heart rate adjusted diastolic delta is calculated according to the formula:
(AoDBP−LVEDP)/HR,
wherein the AoDBP is the aortic diastolic blood pressure, LVEDP is the left ventricular end-diastolic pressure and HR is the heart rate.

14. The method of claim 10 or 11, wherein the heart rate adjusted diastolic delta is calculated according to the formula:
(AoDBP−LVEDP)/HR*80,
wherein the AoDBP is the aortic diastolic blood pressure, LVEDP is the left ventricular end-diastolic pressure and HR is the heart rate and the reference value is about 25.

15. The method of claim 13 or 14, wherein the AoDBP and LVEDP are measured using a device comprising:
a pressure sensor wire assembly for measuring pressure in the heart of a patient, the assembly comprising:
a guide wire, having an aortic section and a ventricle section, the guide wire being insertable through a heart,
an aortic pressure sensor on the aortic section of the guide wire to sense pressure in the aorta;
a ventricle pressure sensor on the ventricle section of the guide wire to sense pressure in the ventricle wherein the distance between the aortic pressure sensor and the ventricular pressure sensor along the guide wire is configured to allow the aortic pressure sensor to be located in the aorta while the ventricle pressure sensor is simultaneously located in the left ventricle; and
an interface on a distal end of the guide wire to send signals from the pressure sensors.

16. The method of claim 13 or 14, wherein the AoDBP and LVEDP are measured simultaneously.
